(19) United States
(12) Patent Application Publication
(10) Pub. No.: US 2011/0251162 A1
(43) Pub. Date: Oct. 13, 2011

(54) METHODS OF TREATMENT FOR MRSA INFECTIONS

(75) Inventors: George Y. Liu, Irvine, CA (US); Ching Wen Tseng, Los Angeles, CA (US)

(73) Assignee: CEDARS-SINAI MEDICAL CENTER, Los Angeles, CA (US)

(21) Appl. No.: 13/120,618

(22) PCT Filed: Sep. 25, 2009
(86) PCT No.: PCT/US2009/058474

§ 371 Date: Mar. 23, 2011

Related U.S. Application Data
(60) Provisional application No. 61/100,233, filed on Sep. 25, 2008.

(51) Int. Cl. A61K 31/56; A61K 31/196; A61K 31/18; A61P 29/00; A61K 31/53; A61K 31/444; A61K 31/40; A61K 31/407; A61K 31/616; A61K 31/381

(52) U.S. Cl. 514/169; 514/165; 514/567; 514/605; 514/448; 514/243; 514/334; 514/423; 514/413

(57) ABSTRACT

The present subject matter relates to pathogenesis of MRSA. Specifically, the present disclosures identifies the pro-inflammatory properties of PVL as the cause of MRSA. Viewed from this new perspective, the present subject matter achieves novel methods and apparatus for treating MRSA infection in a subject involving the administration of an anti-inflammatory drug to the subject. Furthermore the present subject matter teaches methods and apparatus for treating a Panton-Valetine leukocidin associated infection in a subject involving the administration of an anti-inflammatory drug to the subject.

### GOVERNMENT RIGHTS

This subject matter was made with government support provided under Grant No. AI074832 awarded by the National Institutes of Health. The U.S. government has certain rights in the present subject matter.

### FIELD OF THE SUBJECT MATTER

The field of the subject matter relates to methods of treatment for MRSA infections. More specifically, the present subject matter discloses the pro-inflammatory properties of PVL as being the cause of CA-MRSA, and establishes methods for treating CA-MRSA infections by anti-inflammatory agents.

### BACKGROUND OF THE SUBJECT MATTER

All publications herein are incorporated by reference to the same extent as if each individual publication or patent application was specifically and individually indicated to be incorporated by reference. The following description includes information that may be useful in understanding the present subject matter. It is not an admission that any of the information provided herein is prior art or relevant to the presently claimed subject matter, or that any publication specifically or implicitly referenced is prior art.

The present subject matter addresses what is arguably becoming the most important infection of our generation. Despite the intense efforts of the medical community and health administrators, Community Associated MRSA (“CA-MRSA”) has continued to flourish, eluding the understanding of medical experts and averting virtually all efforts at controlling or containing infections.

MRSA is an acronym for methicillin-resistant Staphylococcus aureus (hereafter S. aureus), which is known to produce a variety of toxins and enzymes such as enterotoxin, coagulase and so forth. The coagulase-positive species of S. aureus is well documented as a human opportunistic pathogen [1]. Infections caused by S. aureus are a major cause of morbidity and mortality, especially in hospitals, nursing homes and other care facility settings.

The CA-MRSA epidemic is characterized by an increased incidence of infections, particularly of the skin, and by unusually severe diseases including necrotizing pneumonia, myositis, and necrotizing fasciitis, which were rarely reported prior to the onset of the CA-MRSA epidemic. Of all the factors thought to contribute to the CA-MRSA epidemic, the best known is the Panton-Valentine leukocidin toxin (PVL) which is closely linked epidemiologically with severe human infections. A few carefully performed studies conducted outside the United States (where PVL genes are acquired by both MRSA and methicillin-sensitive staphylococcus aureus alike) constitute the strongest evidence to date that PVL independently determines the severity of skin and lung infections such as furunculosis (spider bite) and necrotizing pneumonia [2, 3]. Because of these largely epidemiologic and clinical reports, PVL has assumed the unproven role of being the most important virulence gene in CA-MRSA, leading to intense investigation of the PVL toxin (403 hits in PubMed since 2000), and increased concern in the lay press: the BBC and other newspapers now commonly refer to CA-MRSA harboring the PVL gene simply as PVL.

Despite high levels of enthusiasm for PVL in the medical and lay press, careful pathogenesis studies of PVL in mice have been strongly conflicting [4, 5, 6]. Despite conclusions to the contrary, the prevailing opinion has now shifted, and concluded PVL to be non-determinant of CA-MRSA disease severity.

The focus of the current subject matter is to address the possible limitations and inconsistencies of previous studies involving the gravity of PVL in defining disease severity, and to identify methods for treatment and containment of CA-MRSA. The results achieved in the present subject matter provides compelling evidence that PVL induces pathology primarily via activation of the host immune system. Accordingly, the present subject matter employs the use of anti-inflammatory agents for effective treatment of CA-MRSA infections and the CA-MRSA epidemic.

### BRIEF DESCRIPTION OF THE FIGURES

Exemplary embodiments are illustrated in referenced figures. It is intended that the embodiments and figures disclosed herein are to be considered illustrative rather than restrictive.

Table 1 identifies strains and plasmids used in the present subject matter.

FIG. 1A(left) depicts a Western blot expression of LukF-PV, LukS-PV, and alpha toxin by CST5+/−2PVL and CST6+/−2PVL. FIG. 1A(right) depicts images of skin and muscle lesions of CD1 mice which were injected subcutaneously with 10⁹ CFU PVL+ S. aureus (Newman+PVL vector, CST5, CST6) on one flank, and 10⁹ CFU isogenic PVL⁻ S. aureus (Newman+empty vector, PVL⁻ CST5, PVL⁻ CST6) on the opposite flank, then sacrificed 3 days post-infection (Arrows point to muscle lesion).

FIG. 1B depicts two graphs representing the skin lesion size of CD1 mice inoculated subcutaneously on both flanks with ˜10⁹ CFU of isogenic PVL-expressing and non-expressing (PVL+/−) strains, sacrificed 3 days post-infection. The graph on the left displays ratio of lesion size based on measurements from each individual mouse. The graph on the right shows lesion size grouped according to bacterial strains.

FIG. 1C depicts two graphs representing the muscle lesion size of CD1 mice inoculated subcutaneously on both flanks with ˜10⁹ CFU of isogenic PVL-expressing and non-expressing (PVL+/−) strains, sacrificed 3 days post-infection. The graph on the left displays ratio of lesion size based on measurements from each individual mouse. The graph on the right shows lesion size grouped according to bacterial strains. (*p, 0.05, **p, 0.01)

FIG. 1D depicts two graphs representing the total tissue CFU of CD1 mice inoculated subcutaneously on both flanks with ˜10⁹ CFU of isogenic PVL-expressing and non-expressing (PVL+/−) strains, sacrificed 3 days post-infection. The graph on the left displays ratio of CFU based on measurements from each individual mouse. The graph on the right shows CFU grouped according to bacterial strains.

FIG. 1E depicts a graph representing the muscle tissue size of CD1 mice which were inoculated subcutaneously on both flanks with ˜10⁹ CFU WT CST5+empty vector, CST5 KO+empty vector, or CST5 KO+PVL expression vector.

FIG. 2A depicts several images of PVL and DAPI stains of the infected tissues of two CD1 mice injected subcutaneously with 10⁹ CFU PVL+ S. aureus (Newman+PVL vector, CST5) on one flank, and 10⁹ CFU isogenic PVL⁻ S. aureus (Newman+empty vector, PVL⁻ CST5) on the opposite flank. PVL immunofluorescence staining of uninfected and infection tissues at 72 h post-infection. Left panels show tissues stained with PVL-FITC Middle panels show DNA was stained with DAPI. Right panels show PVL and DAPI stains merged. (E+D=epidermis-dermis layer; SA=S. aureus; M=muscle)

FIG. 2B depicts several images of PVL immunofluorescence staining of uninfected and infection tissues CD1 mice. Shown are PVL-FITC, phalloidin conjugated to Texas Red, and DAPI (DNA) staining of tissues collected 30 min. post-infection. (E+D=epidermis-dermis layer; SA=S. aureus; M=muscle)

FIG. 2C depicts a graph of the infected CD1 mice in FIG. 2A, providing the level of tissue PVL measured by ELISA at 72 h post-infection, (**p, 0.01)

FIG. 3 depicts four images of the results of H&E staining of day 3 lesions from mice infected with CST5, Newman+empty vector, and Newman+PVL expression vector.

FIG. 4A depicts two graphs for CD1 mice which were injected intraperitoneally with either DPBS, or rabbit antisera against LukS-PV and LukF-PV, and, twenty four hours after initial infection, were infected with Newman +/−PVL bacterial inoculate. Shown are ratios of lesion sizes (PVL⁺:PVL⁻) and muscle lesion sizes from individual mice on day 3 post-infection. (****p, 0.01)

FIG. 4B depicts two graphs for CD1 mice which were injected intraperitoneally with either DPBS, or rabbit antisera against LukS-PV and LukF-PV, and, twenty four hours after initial infection, were infected with CST6+/−PVL bacterial inoculate. Shown are ratios of lesion sizes (PVL⁺:PVL⁻) and muscle lesion sizes from individual mice on day 3 post-infection. (**p, 0.01)

FIGS. 5A-5E depict a series of graphs representing tissue chemokines and cytokines after infection with S. aureus Newman (+/−PVL). CD1 mice were infected on opposite flanks with either Newman+empty vector or Newman+PVL expression vector. Infected tissues were harvested at the indicated time post-infection and tissue chemokines and cytokines were measured by ELISA. (A) Represents Tissue MIP-2; (B) represents RANTES; (C) represents KC; (D) represents IL-1-β and (E) represents TNF-α. Controls consisted of DPBS injected mice. (*p, 0.05)

FIG. 5F depicts a series of graphs representing tissue chemokine and cytokine for MIP-2, KC, and RANTES expressions after infection with isogenic (PVL+/−) S. aureus strains.

FIG. 5G depicts a series of graphs representing tissue chemokine and cytokine for IL-1-α, and TNF-β expression at 8 hours post infection with isogenic (PVL+/−) S. aureus strains.

FIG. 5H depicts a graph representing the effects of innate immunity and host background on PVL virulence function for muscle lesions.

FIG. 5I depicts a graph representing the effects of innate immunity and host background on PVL virulence function for tissue chemokine levels at 0.5 hours and 8 hours after infection with Newman +/−PVL.

FIGS. 6A-6F depict a series of graphs showing the effect of innate immunity and host background on PVL virulence function. Ten to twelve week old CD1, C57BL/6, BALB/c, and SKH1 mice were infected on opposite flanks with either PVL⁺ CST5 or PVL⁻ CST5 S. aureus. Mice were sacrificed at different time points for analyses. (A-D) represent the effect of host background on muscle pathology and total tissue CFU 3 days post-infection with CST5+/−PVL; (A) and (B) plot of muscle lesion sizes and total tissue CFU after infection with WT CST5; (C) and (D) plot of lesion size ratios and CFU ratios (PVL⁺:PVL³¹) from individual mice; (E) and (F) represent the effect of host background on tissue chemokine levels 8 h post-infection with CST5+/−PVL.

FIG. 6G represents the effect of host background on tissue MPO activity 3 h post-infection with CST5+/−PVL. Controls consisted of DPBS injected mice (negative control) and LPS injected mice (positive control). (*p, 0.05, **p, 0.01, ***p, 0.005, ****p, 0.001)

FIGS. 7A-7E depict a series of graphs representing the effect of innate immunity and mouse age on PVL virulence function. Ten to twelve week old CD1 mice were infected on opposite flanks with either PVL⁺ CST5 or isogenic PVL⁻ CST5. (A) represents muscle lesions; (B) represents total tissue CFU 3 days post-infection; (C) represents tissue MPO activity at 3 h postinfection; and (D) and (E) represent KC and MIP-2 levels at 3 h post-infection. (*p, 0.05, **p, 0.01, ***p, 0.005)

FIG. 7F depicts a graph representing tissue MPO levels 3 hours and 12 hours after subcutaneous infection of CD1 mice with CST5 +/−PVL.

FIG. 8A depicts a Western blot showing the immunoblot analyses of overnight bacterial culture supernatant showing LukS-PV, LukF-PV and α-toxin expression by isogenic PVL+/− strain pairs.

FIG. 8B depicts a graph representing the effect of bacterial strains on PVL virulence function in muscle lesions, 3 days after infection with CST5+/−PVL.

FIG. 8C depicts a graph representing the effect of bacterial strains on PVL virulence function in total tissue CFU, 3 days after infection with CST5+/−PVL.

FIG. 8D depicts a graph representing the effect of bacterial strains on PVL virulence function for tissue PVL levels 3 days after infection.

FIG. 9 depicts a graph representing muscle lesion size 3 days after infection with 10⁷ CFU isogenic (PVL+/−) clinical strains, specifically LAC, MW2, SF8300, CST5, and CST6.

### DETAILED DESCRIPTION OF THE SUBJECT MATTER

All references cited herein are incorporated by reference in their entirety as though fully set forth.

Unless defined otherwise, technical and scientific terms used herein have the same meaning as commonly understood by one of ordinary skill in the art to which the subject matter belongs. Singleton et al., Dictionary of Microbiology and Molecular Biology 3rd ed., J. Wiley & Sons (New York, N.Y. 2002); March, Advanced Organic Chemistry Reactions, Mechanisms and Structure 4th ed., J. Wiley & Sons (New York, N.Y. 1992); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present subject matter. Indeed, the present subject matter is in no way limited to the methods and materials described.

“Administering” and/or “Administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods know in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Therapeutically effective amount” as used herein refers to the quantity of a specified compound sufficient to achieve a desired effect in a subject being treated. For example, this can be the amount of an anti-inflammatory drug necessary to prevent, inhibit, reduce, relieve or otherwise treat a condition caused by MRSA infection.

“Treat,” “treating” and “treatment” as used herein refer to both therapeutic treatment and prophylactic or preventative measures, wherein the object is to prevent or slow down (lessen) the targeted pathologic condition, disease or disorder even if the treatment is ultimately unsuccessful. Those in need of treatment may include those already with the disorder as well as those prone to have the disorder or those in whom the disorder is to be prevented.

The present subject matter represents a paradigm shift in the understanding of CA-MRSA pathogenesis. To date, the causative factor responsible for increased disease severity in animals has been attributed to the virulence role of PVL. Accordingly, much of the research and effort directed at identifying CA-MRSA pathogenesis and identifying treatment methods for animals was focused on the cytolytic function of PVL. The present disclosures identify the pro-inflammatory properties of PVL as the leading cause of CA-MRSA. Viewed from this new perspective, the present subject matter provides discloses new methods for treating, preventing and curtailing CA-MRSA in a subject.

Accordingly, the present subject matter provides a method for treating CA-MRSA infection in a subject comprising providing an anti-inflammatory drug and administering the anti-inflammatory drug to the subject by a route of delivery. In various embodiments a manifestation of the CA-MRSA infection treated may include necrotic tissue injury, necrotized fasciitis, muscle inflammation, deep tissue necrosis, necrotizing pneumonia, furunculosis, and combinations thereof.

In another embodiment, the anti-inflammatory drug administered to the CA-MRSA infection may include corticosteroids, cyclooxygenase-inhibitors, ibuprofen, diclofenac, licofelone, aspirin, celecoxib, ketaprofen, piroxicam, sulindac, etoricoxib, fenbufen, fenoprofen, floctafenine, flur-biprofen, tiaprofenic acid, azapropazone, diflunisal, etodolac, lumiracoxib, indomethacin (indometacin), ketorolac, meclofenamate, mefenamic, meloxicam, nabumetone, naproxen, oxaprozin, piroxicam, phenylbutazone, sunlindac, rofecoxib, tenoxicam, tolfenamic acid, tolmetin, valdecoxib, nimesulide, steroids, derivatives thereof, analogs thereof, and combinations thereof.

In a further embodiment, routes of delivery useful in the disclosed methods include but are not limited to non-invasive peroral (through the mouth), topical (skin), transmucosal (nasal, buccal/sublingual, vaginal, ocular and rectal) and inhalation routes, as well as parenteral routes, and other methods know in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

In another embodiment, a therapeutically effective amount of a disclosed anti-inflammatory drug is administered to the subject. The therapeutically effective amount can be determined by one of skill in the art. The effective amount administered to the subject depends on a variety of factors including, but not limited to the age, body weight, general health, sex and diet of the subject being treated, the condition being treated, the severity of the condition, the activity of the specific anti-inflammatory drug being administered, the metabolic stability and length of action of that drug, mode and time of administration, rate of excretion and the drug combination. The amount of the pharmaceutical composition that is effective in the treatment or prevention of a condition, such as a MRSA infection, can be determined by standard clinical techniques well known to those of skill in the art. In addition, in vitro or in vivo assays can optionally be employed to help identify optimal dosage ranges. One of ordinary skill in the art will readily be able determine the precise dose to be employed.

In addition the present subject matter further discloses a method for treating a Panton-Valetine leukocidin associated infection in a subject comprising providing an anti-inflammatory drug and administering the anti-inflammatory drug to the subject by a route of delivery. In various embodiments a manifestation of the Panton-Valetine leukocidin associated infection treated may include necrotic tissue injury, necrotized fasciitis, muscle inflammation, deep tissue necrosis, necrotizing pneumonia, furunculosis, and combinations thereof.

In another embodiment, the anti-inflammatory drug administered to the Panton-Valetine leukocidin associated infection may include corticosteroids, cyclooxygenase-inhibitors, ibuprofen, diclofenac, licofelone, aspirin, celecoxib, ketaprofen, piroxicam, sulindac, etoricoxib, fenbufen, fenoprofen, floctafenine, flurbiprofen, tiaprofenic acid, azapropazone, diflunisal, etodolac, lumiracoxib, indomethacin (indometacin), ketorolac, meclofenamate, mefenamic, meloxicam, nabumetone, naproxen, oxaprozin, piroxicam, phenylbutazone, sunlindac, rofecoxib, tenoxicam, tolfenamic acid, tolmetin, valdecoxib, nimesulide, steroids, derivatives thereof, analogs thereof, and combinations thereof.

In a further embodiment the routes of delivery are non-invasive peroral (through the mouth), topical (skin), transmucosal (nasal, buccal/sublingual, vaginal, ocular and rectal) and inhalation routes, as well as parenteral routes, and other methods know in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

In another embodiment, a therapeutically effective amount of a disclosed anti-inflammatory drug is administered to the subject. The therapeutically effective amount can be determined by one of skill in the art. The effective amount administered to the subject depends on a variety of factors including, but not limited to the age, body weight, general health, sex and diet of the subject being treated, the condition being treated, the severity of the condition, the activity of the specific anti-inflammatory drug being administered, the metabolic stability and length of action of that drug, mode and time of administration, rate of excretion and the drug combination. The amount of the pharmaceutical composition that is effective in the treatment or prevention of a condition, such as a Panton-Valetine leukocidin associated infection, can be determined by standard clinical techniques well known to those of skill in the art. In addition, in vitro or in vivo assays can optionally be employed to help identify optimal dosage ranges. One of ordinary skill in the art will readily be able determine the precise dose to be employed.

Multiple epidemiologic studies have provided compelling evidence linking PVL to pathogenicity of S. aureus infections [8, 9, 26-28]. Notably, an S. aureus strain responsible for an epidemic in the 1950's (phage type 81/80) also harbored the LukFS-PV genes; the strain mysteriously disappeared following the introduction of methicillin [29], but PVL resurfaced in the 1990's in association with severe cases of necrotizing pneumonia and furunculosis in MSSA (Methicillin-Sensitive S. aureus) and CA-MRSA strains [2, 30]. However, whether PVL itself is a causative factor responsible for increased disease severity has been heatedly debated because of the failure of multiple groups to demonstrate that PVL has a virulence role [4-6, 31-33]. More specifically, in the skin infection model, Voyich et al. and subsequently Bubeck et al. infected C57BL/6 and BALB/c mice with 10⁷ WT or PVL KO in the S. aureus LAC USA300 background. In both studies, the authors report either no difference in skin lesion size (in BALB/c or C57BL/6 mice) or a larger skin lesion (in BALB/c AnNHsd mice) inoculated with the PVL mutant strain. The authors did not evaluate muscle lesions in these studies. Brown and coworkers performed an infection experiment using the same inoculum and bacterial strains, and reported a visual difference in muscle lesion size on day 7, albeit the lesion sizes were not quantitated [32]. Here, in a model of severe soft tissue infection achieved using a higher S. aureus inoculum, we show that PVL contributes significantly to the severity of muscle tissue pathology.

Szmigielski and coworkers have reported that mouse phagocytes are less susceptible to lysis compared to human phagocytes and show an approximate ten fold difference in membrane permeability following purified PVL toxin challenge as measured by 51-Cr release assay [34]. Additionally, the present subject matter shows that the concentration of PVL measured in mouse tissue, following subcutaneous infection with 10⁹ CFU PVL⁺ CA-MRSA, is in the range of 10-20 μg/ml, which even with an underestimate of the PVL concentration in infected tissue for technical reasons, is 50-100 fold lower than PVL measured from human abscess samples (median, 1 μg/ml) [35]. The possibility of differential PVL induction in human and mouse tissues in human and mouse response may be a confounding factor in extrapolating animal data to the human condition. Accordingly, the role of PVL in human diseases will need to be addressed by the use of humanized animals, or possibly by the therapeutic effect of PVL-specific antibodies on human CA-MRSA infections.

Notwithstanding the differences between human and mouse infection, the present subject matter has features that mimic the human disease. PVL in the mouse preferentially causes injury of the underlying muscle following subcutaneous CA-MRSA injection, and histologic evaluation showed moderate staining of muscle tissues with anti-PVL antibodies. Consistent with this finding, we have recently submitted a report of a child with myositis caused by a PVL⁺ methicillin-sensitive S. aureus strain, whose muscle tissue stained selectively and strongly with a PVL specific antibody. It is not known how PVL binding to muscle tissues or PVL induced chemokines contribute to muscle injury, however, given the reported association of severe myositis with CA-MRSA and PVL, the parallel findings in human and mouse strongly suggest a causal link between PVL and human myositis [36].

Multiple studies point to a strong association between PVL and severe necrotizing pneumonia and furunculosis in the current CA-MRSA epidemic [1, 2]. These infections appear to target young and healthy hosts, who appear to suffer more severe infection compared to older or immunosuppressed individuals. Consistent with these findings, we observed that PVL induced greater inflammation and caused greater pathology in mice without conferring a bacterial survival advantage. Further, a PVL effect on tissue pathology was apparent in those mice with the most effective immune clearance of S. aureus (10-12 week old CD1 and BALB/c mice), but was insignificant in mice that had a more limited response to the pathogen (C57BL/6, SKH1, and 6 month old CD1 mice). These findings, when taken together with the cytokine results, provide a parallel between the present mouse model and human infection, and suggest two conclusions: 1) in CA-MRSA, a primary pathogenic effect of PVL is to provoke an overly-robust inflammation; and 2) the severity of PVL-specific pathology may ultimately depend on the capacity of the host immune system for mounting an aggressive neutrophil response to infection. These results are consistent with human epidemiologic reports indicating that younger, immunocompetent individuals infected with MRSA are more susceptible to severe injury [1, 2], and suggest that individuals with enhanced immune response to pathogens are at higher risk for more serious pathology.

Prior efforts to link PVL to human pathology have focused primarily on the cytolytic properties of PVL. Lysis of human phagocytes could be readily demonstrated using purified PVL [34], but a study conducted using live WT and PVL knockout S. aureus showed no lytic effect of the toxin on human neutrophils across a range of bacterial concentrations [5]. The present subject matter provides that a function of PVL more readily achievable at physiologic doses is its ability to provoke inflammation and recruit immune effector cells such as neutrophils through activation of inflammatory molecules (e.g., RANTES, KC, and MIP-2). In vitro, PVL induction of IL8 could be demonstrated using human neutrophils at a concentration lower than that required to induce cell lysis [37]. These findings suggest that PVL induced PMN cytolysis as the primary explanation of PVL injury may need to be reevaluated. Under the present subject matter, enhanced inflammation, particularly recruitment of phagocytes, could explain PVL-associated “spider bite” lesions and abscesses (representing phagocyte accumulation), which have become the most common presentation of CA-MRSA skin and soft tissue infections [38]. Though the present application highlights differences in chemokine induction by PVL⁺ and PVL⁻ strains, PVL shows to induce additional pro-inflammatory factors during infection. In tissue culture, Konig and coworkers have shown that PVL triggers secretion of leukotriene B4 and oxygen metabolites when incubated in the presence of human neutrophils [37]. Hence, inflammation initiated by multiple proinflammatory mediators is likely a pivotal contributor to PVL mediated pathology.

Further, epidemiologic studies have shown compelling evidence linking PVL to pathogenicity of the MRSA epidemic, however experiments conducted on mice using genetically modified CA-MRSA strains have failed to confirm a central role for PVL in determining the severity of the disease [7, 8, 9]. The results revealed in the present subject matter indicate that this apparent failure is due to insensitivity of the mouse infection model relative to human infections. PVL caused necrotic tissue injury of the underlying muscle, but the presence or absence of PVL fails to impact skin lesion severity. Our findings show that expression of PVL by necrotizing fasciitis isolates can lead to more severe pathology not readily apparent on cutaneous inspection in mice. These findings further corroborate the plethora of epidemiologic reports that implicate PVL in severe infections associated with muscle inflammation and deep tissue necrosis [10, 11]. Further validating the finding of the present subject matter.

Accordingly, the present subject matter concludes that application of antibodies against PVL could limit or even abrogate PVL-mediated injury. In preliminary experiments, we have tested few additional CA-MRSA WT and PVL KO strains (LAC and MW2), but found that under our experimental conditions, PVL expressed by LAC and MW2 was associated with increased bacterial survival but similar level of muscle tissue injury on day 3 (unpublished data).

In summary, the present subject matter has established a model of severe necrotizing soft tissue infection in which PVL shows significant contribution to muscle injury. Though the model does not by itself resolve the debate on the relative importance of PVL in the MRSA epidemic, it unveils surprising parallels between the mouse and human disease, and provides novel insights towards PVL related immunopathology, and methods for effectively treating MRSA.

### Methods

### Bacterial Strains and Growth Conditions

Five clinical CA-MRSA isolates (LAC, MW2, SF8300, CST5, and CST6) and their isogenic PVL knockout strains were used for this study (Table 1). The construction of isogenic PVL knockout strains in the LAC and MW2 backgrounds have been described herein. PVL knockout in the SF8300 background was engineered as described for LAC and MW2 PVL knockout, with a spectinomycin resistance cassette replacing both the lukS-PV and lukF-PV genes. The PVL knockout of CST5 and CST6 isolates were constructed by site-directed mutagenesis using primer pairs SEQ. ID. NO. 1, SEQ. ID. NO. 2, SEQ. ID. NO, 3, and SEQ. ID. NO. 4 to introduce two stop codons in the lukS-PV open reading frame (ORF). For complementation and overexpression studies, the PVL locus was amplified by PCR using primer pair SEQ. ID. NO. 5, and SEQ. ID. NO. 6 and cloned into shuttle vectors (Table 1). Bacteria were cultured in Todd-Hewitt broth or L-broth at 37° C. with shaking at 250 rpm.

### Generation of Rabbit Antisera

Rabbits were hyperimmunized with recombinant PVL proteins in emulsion with Freund's adjuvant to generate specific, high-titer antisera to both LukS-PV and LukF-PV. For each protein, two rabbits were immunized. After 4 immunizations (on days 0, 7, 21, and 35) specific antibody titers increased 500,000-fold for both rabbits immunized with rLukF-PV and >2,000,000-fold for one rabbit immunized with rLukS-PV.

Cloning and Expression of rLukF-PV and rLukS-PV

lukS-PV and lukF-PV were amplified by PCR using flanking primer sequences (lukS-PV: SEQ. ID. NO. 7 and SEQ. ID. NO. 8; lukF-PV SEQ. ID. NO. 9, and SEQ. ID. NO. 10) and cloned into pET151/TOPO-D (Invitrogen). Recombinant PVL proteins were expressed in E. coil at 30° C. in the presence of 1 mM IPTG, His-6 tagged proteins were purified over nickel/cadmium columns, and quantitated by BCA. The His-6 tag was removed by AcTEV protease (Invitrogen), with cleavage confirmed by SDS-PAGE.

### Murine Skin Infection Model

Ten week old SKH1, CD1, and BALB/c mice and 6 month old CD1 mice were purchased from Charles River Laboratories. C57BL/6 mice were obtained from The Jackson Laboratory. Overnight bacterial culture was diluted 1:1000 in prewarmed media and incubated at 37° C. with shaking at 250 rpm until an A₅₄₀˜2.5. Bacteria were harvested by centrifugation at 4000 rpm for 10 minutes at 4° C., and then washed twice with equal volume of DPBS (Mediatech). The bacteria were resuspended in DPBS at a concentration of 10⁸-10¹⁰ CFU/mL (±20%), and 100 mL of suspension were injected subcutaneously in each flank. Injections were performed with careful visualization of the needle to assure that the injection is not intramuscular.

For experiments assessing the therapeutic efficacy of anti-PVL antibodies, 1 mL of rabbit antisera against LukS-PV and LukFPV, or DPBS were administered intraperitoneally at 24 h prior to infection.

### Determination of Lesion Size and Tissue Bacterial CFUs

Following euthanization, skin and muscle lesions were measured and both tissues were excised separately and homogenized in 1 mL of DPBS for CFU determination. The homogenized suspension was centrifuged at 15,000×g for 10 min and supernatants were stored at −80° C. for subsequent analysis by ELISA. For the skin, lesions were defined by darkened areas of necrosis; for muscle, the lesions consisted of raised pale or darkened colored lesions overlying the red colored healthy tissue. An area of hyperemia is often visualized around the muscle lesions. Muscle lesions are further differentiated from occasional fat tissue based on color and consistency.

Our method to measure lesion size has been previously reported [39]. Both skin and muscle lesions were quantitated by multiplying the length and width of the lesion. Irregularly-shaped lesions were broken down into smaller symmetrical pieces, and each piece was measured by the same method.

To further evaluate this methodology, we assessed retest reproducibility (i.e., the intra-observer coefficient of variability [CV]), inter-observer variability, and calculated intra-class correlation coefficients (ICCs) [40] in a subset of lesions (n=20; half were PVL and half were PVL⁻, and lesions spanned a wide range) using two blinded observers. We also performed lesion measurements using an independent measurement method that utilized computer-assisted histomorphometric assessment of lesion area (ImageJ; open-source available from the NIH at http://rsb.info.nih.gov/ij/) [41]. Two blinded investigators with experience in mouse anatomy and lesion measurement independently assessed twenty lesions spanning a wide range of lesion sizes using the manual method (i.e., measurement of lesion length and width) and the computer-assisted histomorphometric method (i.e., ImageJ). Each observer then measured each lesion a second time after the lesions were shuffled to control for order effects. Lesion sizes were expressed as both area measurements (in mm²) and as a dimensionless ratio of left (i.e., PVL) to right (i.e., PVL⁻). Intraobserver CVs (i.e., retest reproducibility) were between 6% and 11%, inter-observer CVs were 6% to 12%. ICCs were 0.882 for the manual method and 0.960 for the histomorphometric technique. The overall ICC between the two methods was 0.930.

### Enzyme Linked Immunosorbent Assay (ELISA)

Mouse MIP-2, RANTES, TNF-α, IL-1β, and KC (R & D Systems) specific ELISAs were performed according to the manufacturer's instructions. For PVL ELISA, plates were coated overnight at 4° C. with known concentrations of rLukF-PV or test samples. The wells were blocked using 5% skim milk and 0.5% normal goat serum (Sigma) for an hour at 37° C. Rabbit anti-LukFPVL anti-serum (1:20,000 in the blocking buffer) was added to each well, After 1 hour at 37° C., the wells were washed 3 times with PBS plus 0.1% Tween-20 (wash buffer). A goat anti-rabbit IgG conjugated to HRP (Cell Signaling) (1:5,000 in blocking buffer) was next added for an hour at 37° C., and after 5 washes, HRP activity was detected using TMB substrate (Fisher Scientific). Threshold detection level of the assay is 2 μg/mL. PVL ELISA has been validated using supernatant from mouse tissue infected with PVL⁻ S. aureus, spiked with known concentrations of rLukF-PV toxin.

### Immunofluorescent Assay (IFA), and Hematoxylin-Eosin (H&E) Stain

For IFA, the infected tissue was excised and fixed in 10% formalin (Medical Chemical Corporation) overnight. Paraffin embedding and H&E staining were performed by the Department of Pathology at Cedars-Sinai Medical Center. For IFA, tissue sections were deparaffinized and blocked with 5% goat serum in PBS with 0.05% Tween 20 (ISC Biosciences) (blocking buffer) for 1 hour at 37° C. After incubating samples with rabbit anti-LukSPV antibody (diluted at 1:200 in blocking buffer) at 37° C. for 1 hour, slides were washed 5 min with PBS 3 times and incubated with corresponding FITC-conjugated secondary antibody (Sigma). One unit of Texas Red-phalloidin (Invitrogen) was used for ounter staining per slide. After a final wash, tissue sections were counted with Prolong AntiFade containing DAPI (Invitrogen). A minimum of six mice were used for each condition. Stained slides were examined using Olympus BX51 fluoresces microscope.

### Myeloperoxidase (MPO) Assay

Animals were euthanized at 3 hours or 12 hours post-infection, and the skin and muscle lesions were homogenized in 1 mL of DPBS-Tx100 (0.5%) with protease inhibitor cocktails (Roche). The homogenized suspension was centrifuged at 15,000×g for 10 min. and supernatants were collected and assayed for MPO activity according to the manufacturer's instructions (Invitrogen). A minimum of six mice were used for each condition.

### Immunoblot Analysis

Overnight bacterial cultures were standardized by % T₅₄₀ to a concentration of 10⁹ CFU/mL. The supernatants were collected by centrifugation and separated using Nu-PAGE system (Invitrogen). Proteins were blotted onto nitrocellulose membranes and probed with specific antibodies against LukS-PV, LukF-PV, and α-toxin (Sigma) and corresponding secondary antibodies conjugated to horseradish peroxidase (Cell Signaling Technology Inc.). Specific proteins were visualized using ECLplus (Amersham Biosciences).

### Statistical Analysis

Data were analyzed using Prism 4.03 (Graphpad Software, Inc.). The two-tailed Wilcoxon test was used to compare paired samples. Unpaired samples were analyzed using Mann-Whitney test. For between group comparisons, the Kruskal-Wallis test was used. Unless otherwise indicated, a p value less than 0.05 was considered significant, and noted in the figures. In bar graphs, n≧6 and results are presented as mean±SEM.

### Results

### PVL Contributes to Muscle but not Skin Injury

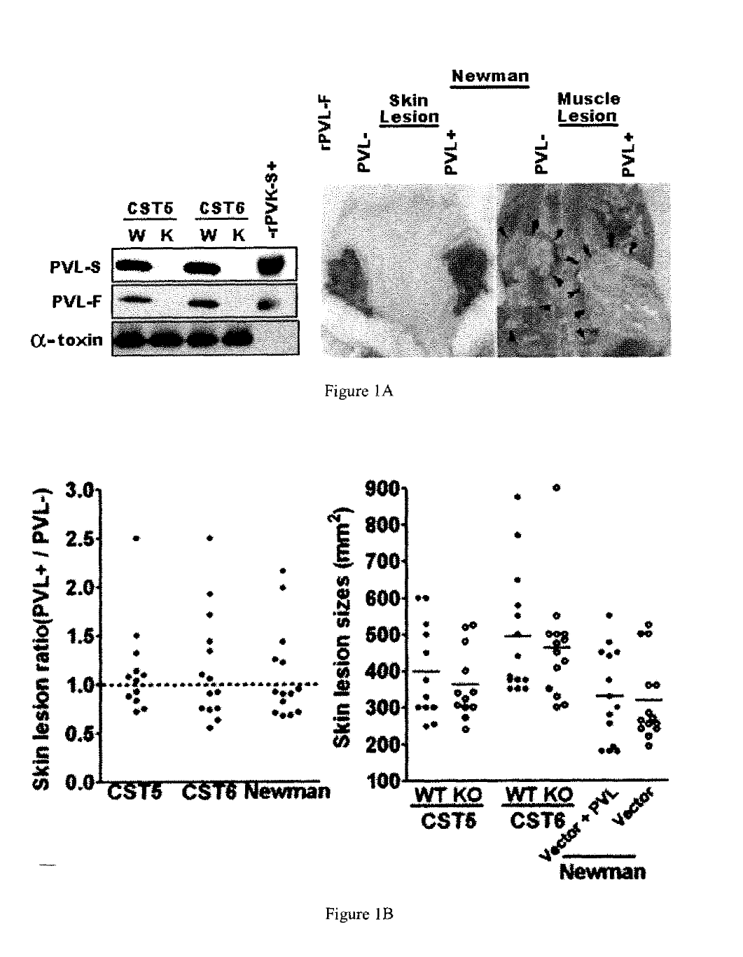

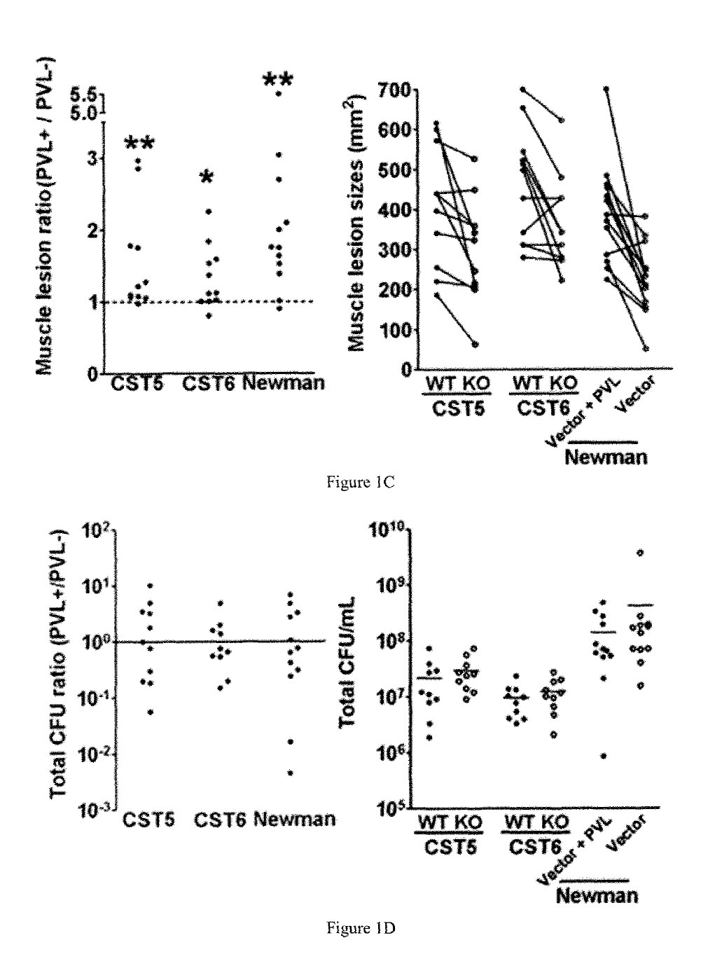

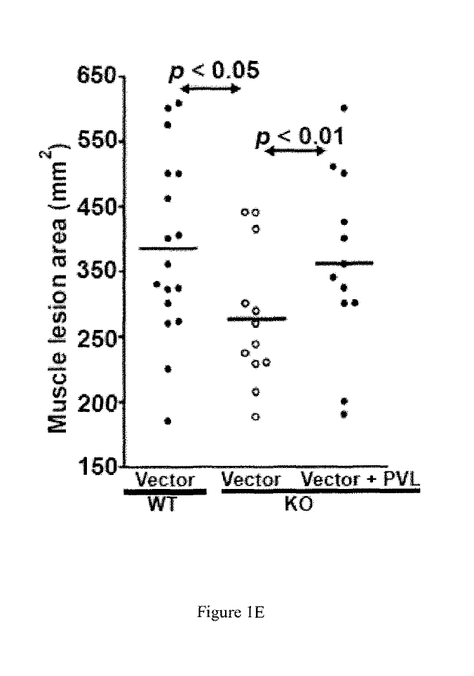

PVL virulence studies were performed in a murine model of severe soft tissue infection designed to mimic human necrotizing fasciitis/myositis. Bacterial strains tested in this infection model included two PVL⁺ USA300 strains isolated from wounds of patients with necrotizing fasciitis (CST5 and CST6), a PVL⁻ strain (Newman), and PVL⁺ or PVL⁻ isogenic strains engineered from these bacteria [10] (Table 1). A Western blot confirming PVL expression by PVL strains, but not PVL⁻ strains, is shown in FIG. 1A(left). Previous investigations have demonstrated that a minimum

CD1 mice were inoculated subcutaneously on both flanks with 10⁹ CFU of either isogenic PVL⁺ strain or PVL⁻ strain and were sacrificed 3 days after infection for analyses of lesions. As shown in FIGS. 1A and 1B, no differences in skin lesion size were observed in mice infected with PVL⁺ and PVL⁻ strains. However, PVL⁺ strains induced larger muscle lesions compared to isogenic PVL⁻ strains. Complementation with a PVL expression vector restored the ability of the PVL mutant to cause more severe muscle injury (FIG. 1E). Surprisingly, the numbers of bacteria recovered from lesions produced by PVL⁺ and PVL⁻ bacteria were comparable (FIG. 1D and FIG. 1E), hence the increased lesion severity associated with PVL⁺ strains could not be explained by increased bacterial survival in viva CD1 mice, infected with 107 or 108 PVL⁺ or PVL⁻ isogenic strains, showed no difference in muscle tissue injury (FIG. 1C).

### Histology and PVL Expression in Infected Tissue

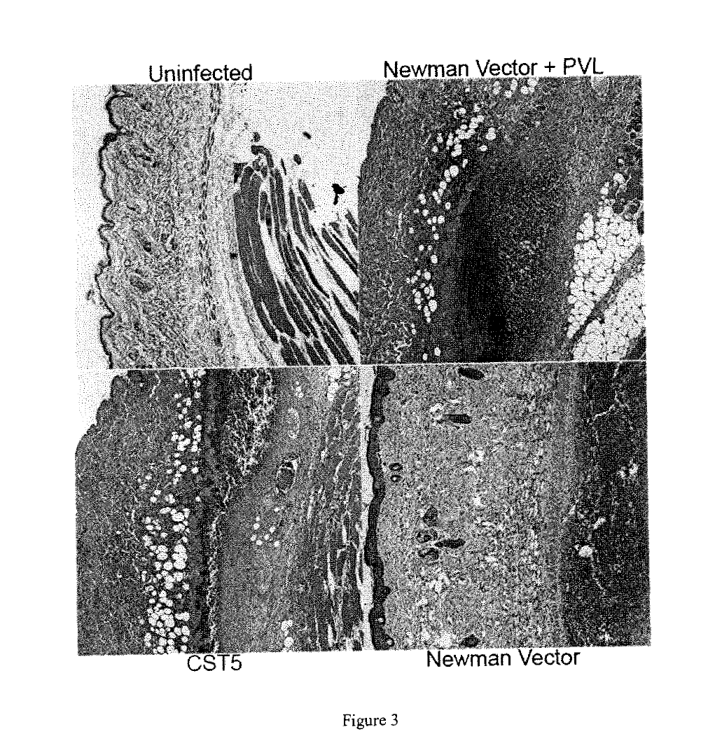

To determine whether PVL⁺ and PVL⁻ strains contribute to differences in tissue histology, H&E stain was performed. Overall, H&E stain showed marked necrosis and neutrophil infiltration within the central focus of infection produced by PVL⁺ or PVL⁻ strains, but beyond lesion size differences, a clear-cut difference in pathology was not discernable (FIG. 3).

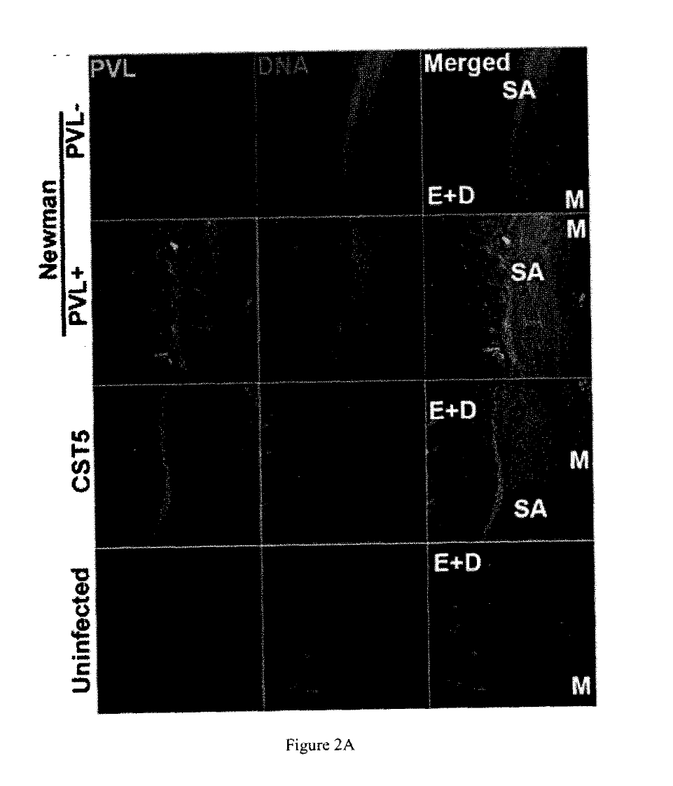

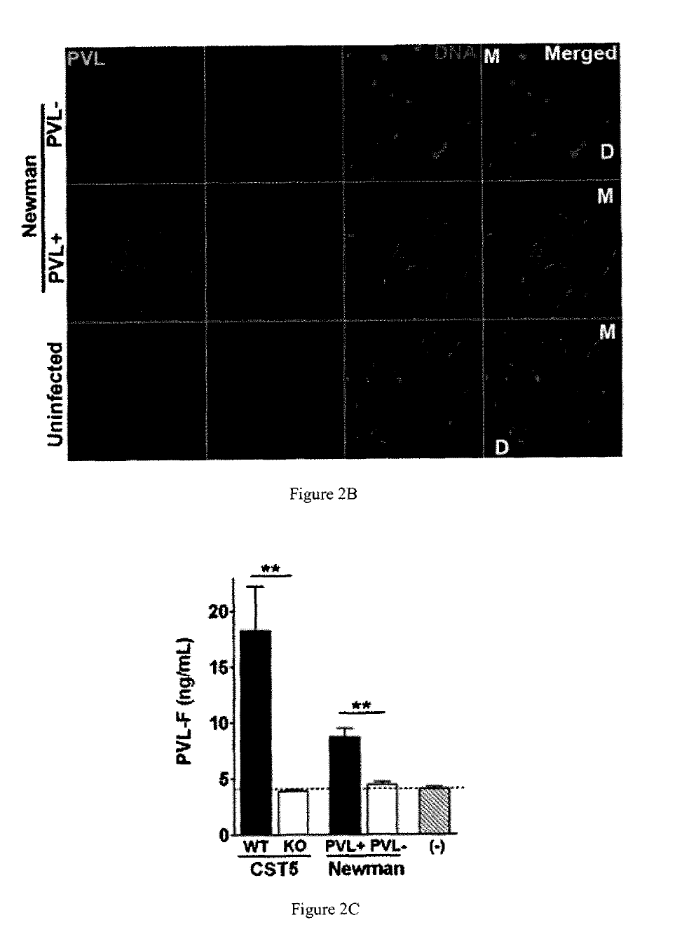

To examine the PVL contribution to pathology in more detail, infected tissues were excised for histological analysis and PVL detection by immunohistochemistry. As evidenced by diffuse staining throughout the tissue slices, the PVL⁺ necrotizing fasciitis clinical isolate (CST5) and PVL⁺ Newman strain both expressed the toxin in vivo (FIG. 2A). PVL staining was particularly prominent around PVL⁺ S. aureus clusters, but was also noted on select muscle bundles, particularly at early time points of infection (FIGS. 2A and B). Based on measurement of PVL concentration by ELISA (FIG. 2C), PVL⁺ Newman secretes lower levels of PVL compared to CST5, even though Newman showed a more prominent PVL effect on muscle injury compared to CST5. The measured toxin level in tissues was in the range of 10-20 ng/ml at day 3, and never significantly exceeded those levels at earlier time points based on a time course experiment performed using PVL⁺ Newman (data not shown). Of note, the ELISA is likely to underestimate the actual PVL concentration in infected tissue since much of the toxin that intercalated into host cell membranes might not be have been adequately solubilized to permit measurement in an ELISA.

### PVL Antibodies Block PVL Induced Muscle Injury

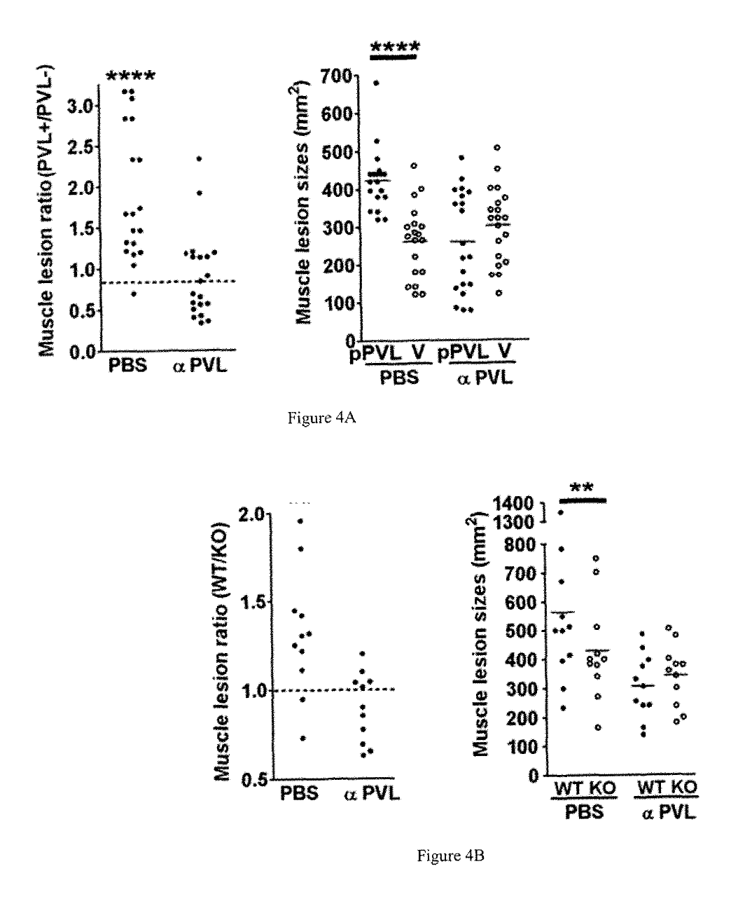

To determine whether blocking PVL could reduce the extent of muscle injury, mice were injected intraperitoneally with PVL-specific antibodies or PBS, then challenged the next day with 10⁹ bacteria. As shown in FIG. 4A and FIG. 4B, pretreatment with PVL-specific antibodies significantly reduced the size of muscle lesions induced by PVL⁺ strains, but did not alter lesion sizes caused by PVL⁻ strains. These experiments establish a pathogenic role of PVL in severe soft tissue infections in mice, and indicate that damage inflicted by PVL predominantly affects deep muscle tissues and not superficial skin layers.

### Contribution of PVL to Inflammation

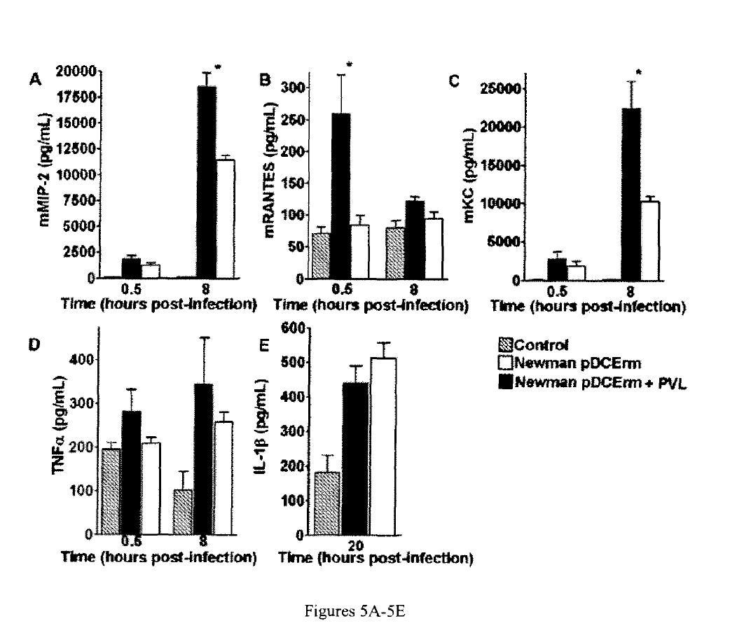

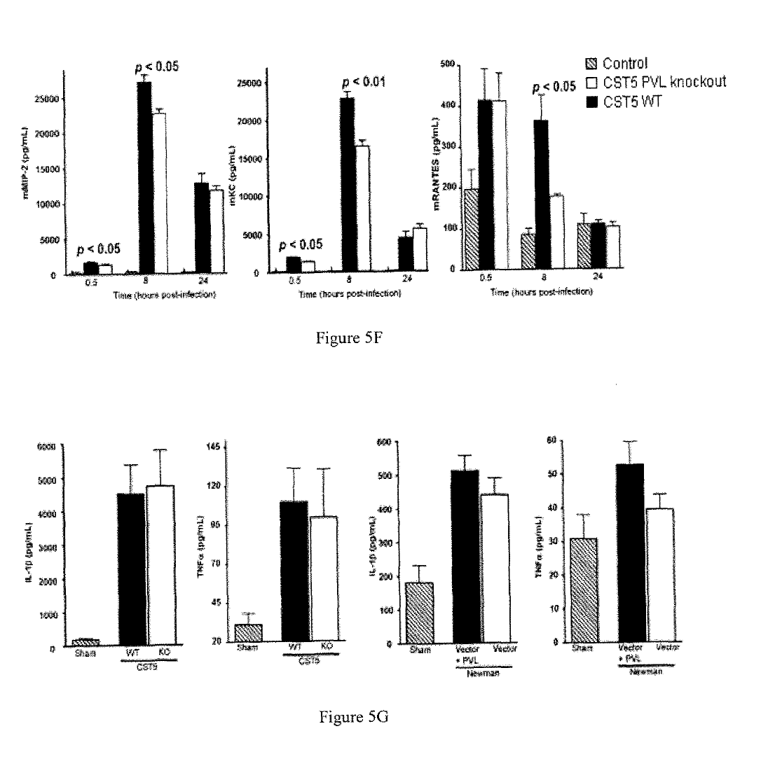

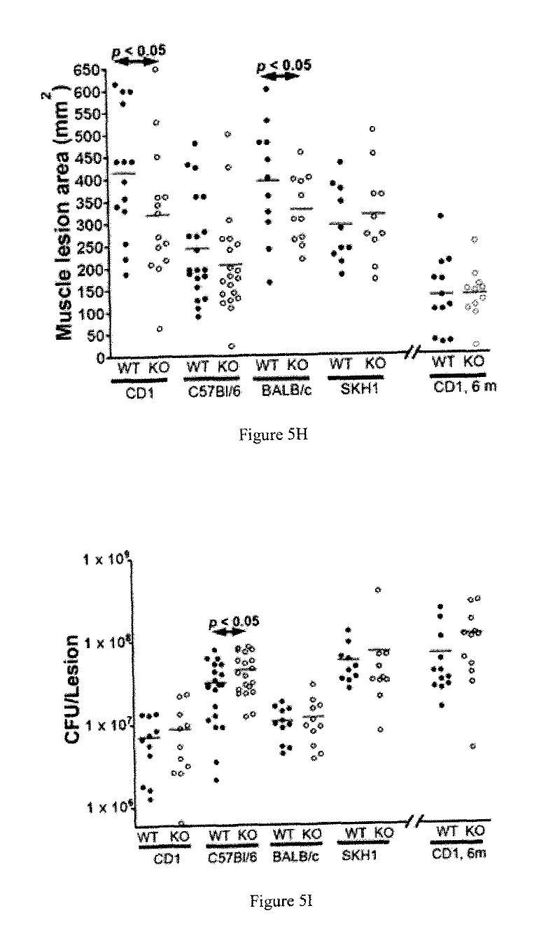

It is well established that host inflammation can have devastating consequences during infection [13]. In our necrotizing soft tissue infection model, damage to muscle tissues could be inflicted by direct effects (e.g., cytolytic activity) of PVL, indirectly by the host response to the toxin, or both in combination. Direct cytolytic activity of the toxin has been difficult to demonstrate using WT and isogenic PVL knockout strains, even when human neutrophils are used as target cells [5]. To investigate how PVL might affect inflammation, lesions were isolated from infected CD1 mice and expression of cytokines and chemokines measured by ELISA. As shown in FIGS. 5A-5G, both PVL expressing Newman and CST5 strains induced higher levels of the CXC family chemokines KC, MIP-2, and RANTES in the injured tissues, but no significant differences in TNF-α and IL1-β were observed between the two strains. These data are in agreement with prior finding that low non-cytolytic dose of purified toxins induced human CXC-family chemokine IL1-β secretion by neutrophils in vitro [16].

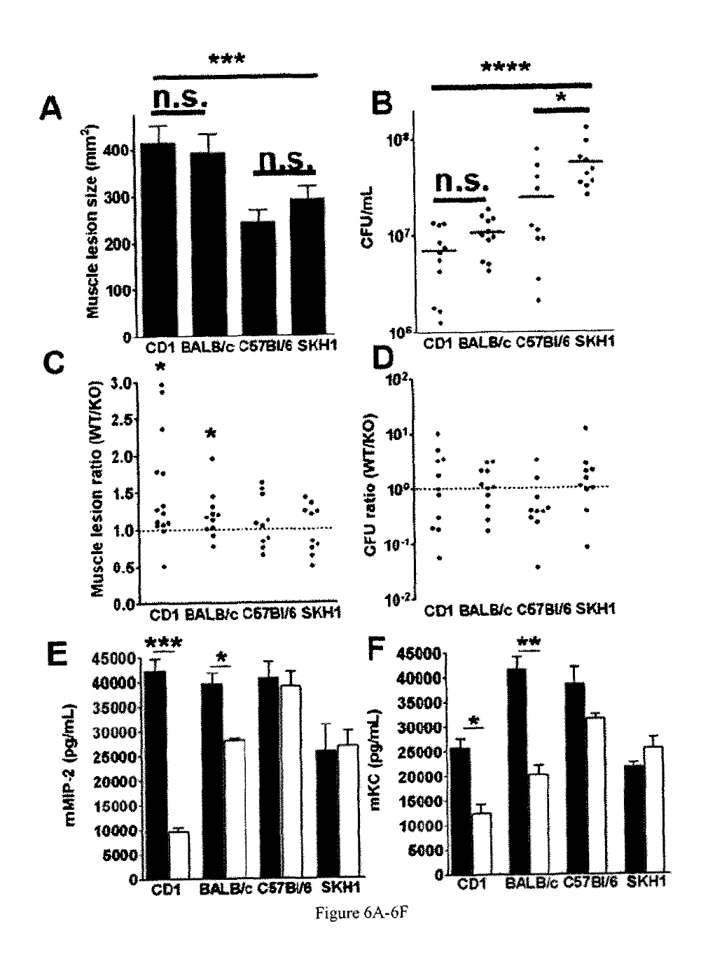

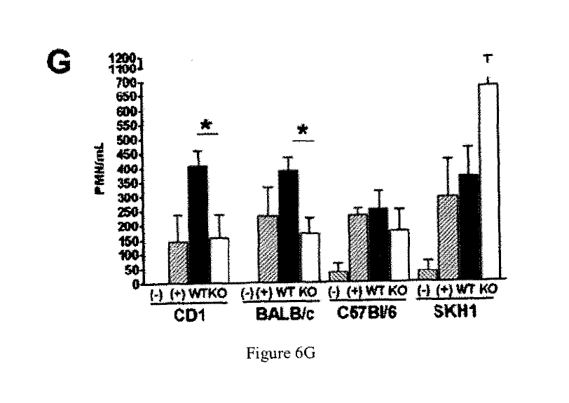

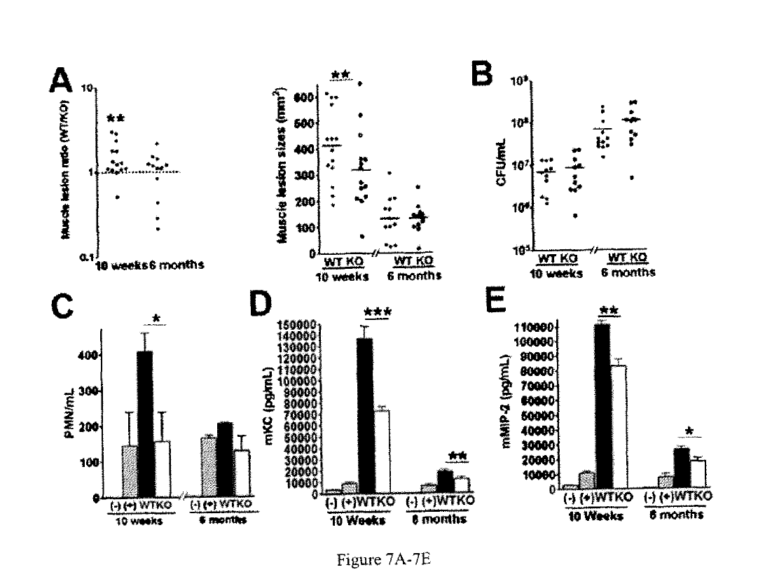

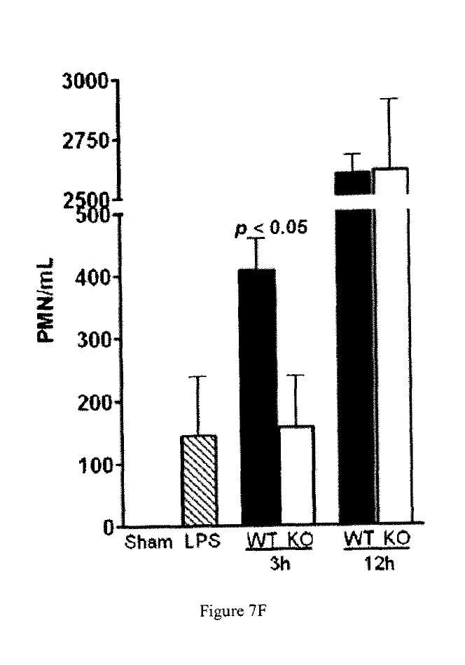

Previous investigations of PVL virulence functions identified conflicting roles of the toxin in S. aureus pathogenesis, but a variable in the prior studies was the use of three different inbred mouse strains, namely BALB/C, C57BL/6 and the hairless SKH1 [4, 5, 6]. Interestingly, in a model of Chlamydia infection, BALB/C mice secreted higher levels of chemokines and had more severe pathology compared to C57BL/6 mice [17], consistent with prior findings that inbred strains of mice exhibit marked differences in host defensive responses [18]. We therefore hypothesized if a central function of PVL is induction of inflammation, then the impact of PVL on disease severity could importantly depend on the magnitude of the immune response mounted by the host. To test this hypothesis, we examined skin and soft tissue infection in all three mouse strains used in previous PVL studies (BALB/c, C57BL/6, SKH1), as well as the outbred strain CD1. As shown in FIGS. 5H and 5I, following infection with WT PVL⁺ S. aureus CST5, CD1 and BALB/c mice developed significantly larger muscle lesions compared to SKH1 or C57BL/6 mice, but had fewer numbers of recovered bacteria. These results suggest that CD1 and BALB/C mice had more effective immune clearance of bacteria, but suffered more severe injury secondary to excessive inflammation. Strikingly, PVL contributed markedly to severe muscle lesions in these same two mouse strains, but in SKH1 and C57BL/6 mice that had a more limited response to S. aureus challenge, PVL had no effect on severity of muscle lesions. Given the ability of PVL to induce chemokines in outbred CD1 mice, we next asked whether PVL induced chemokine secretion is associated with increased lesion sizes in the different mouse strains. As shown in FIGS. 6E and 6F, the CST5 PVL⁺ strain elicited significantly higher levels of MIP-2 and KC in CD1 and BALB/c mice compared with the isogenic PVL⁻ strain, but this chemokine differential did not occur in SKH1 or C57BL/6 mice. Furthermore, PVL induced a significant increase in neutrophil infiltration as measured by tissue myeloperoxidase (MPO) activity in CD1 and BALB/c mice at 3 hours, but not in SKH1 and C57BL/6 mice (FIG. 6G). No difference was seen between WT S. aureus and the isogenic PVL knockout mutant when MPO was measured at 12 hours (FIG. 7F).

Overall, these experiments indicate that PVL activates host immune responses to variable degrees depending on the genetic background of the host, and that the robustness of the host response is a determinant of the severity of PVL-associated deep tissue injury.

### Effect of Host Genetic Background on PVL Virulence

Next, to facilitate studies of immune response to PVL, we repeated the infection experiments in C57/B6 mice, the background mouse strain on which most knockouts are maintained. Injection of C57/B6 mice with 10⁹ CFU WT CST5 and PVL CST5 mutant, unexpectedly, elicited muscle lesions of comparable size (FIG. 6C), suggesting that the mouse's genetic background is a further determinant of PVL-induced disease pathology. Previously, Bubeck and colleagues have demonstrate that host background differences could be a determinant of PVL virulence [4, 31]. In their study, PVL showed no virulence effect in C57BL/6 mice [4] but paradoxically attenuated pathogenicity of S. aureus in BALB/c AnNHsd mice [31]. To further evaluate whether and how the mouse genetic background and immune system contribute to PVL mediated injury, we examined skin and soft tissue infection in four different mouse strains: BALB/c, C57BL/6, SKH1, and CD1. As shown in FIG. 6C, PVL contributed to muscle lesions in CD1 and BALB/c mice, but had no effect on lesion severity in SKH1 and C57BL/6 mice. The presence of PVL related injury in any particular strain of mouse correlated directly to differences in PVL associated chemokine secretion (FIGS. 6E and 6F). Specifically, the CST5 PVL⁺ strain elicited significantly higher levels of MIP-2 and KC in CD1 and BALB/c mice compared with the isogenic PVL⁻ strain, but this chemokine differential did not occur in SKH1 or C57BL/6 mice. Furthermore, as measured by tissue MPO activity, PVL induced increased neutrophil infiltration in CD1 and (to a lesser extent) in BALB/c mice at 3 h post-infection, but not in SKH1 and C57BL/6 mice (FIG. 6G). No difference was observed between WT S. aureus and the isogenic PVL knockout mutant when MPO was measured at 12 h (FIG. 7F). Overall, these results suggest that PVL induction of host immune responses depends on the genetic background of the mouse. We noted interestingly that CD1 and BALB/c mice (+PVL phenotype) cleared WT CST5 infection much more effectively compared to SKH1 or C57BL/6 mice (no PVL phenotype), but developed significantly larger lesions compared to SKH1 or C57BL/6 mice (FIGS. 6A and 6B). A possible interpretation of these findings could be that CD1 and BALB/c mice detect and respond to PVL with an exaggerated proinflammatory reaction and neutrophil recruitment, which achieves more rapid bacterial clearance, but at a simultaneous cost of more extensive collateral damage to host tissues.

### Pathogenicity of PVL in Older Mice

Overly exuberant immune response has been shown to contribute to more severe pathology in young and previously healthy victims of the 1918 influenza epidemic [12]. Interestingly, multiple CA-MRSA epidemiologic studies have also linked PVL to more severe infections among young and healthy individuals [2, 3]. We therefore reasoned that young mice might mount a more robust immune defense against PVL⁺ MRSA strains compared to older mice from the same background strain. To directly test this hypothesis, 6 month old mice were injected subcutaneously with isogenic PVL⁺/⁻ CST5 strains. As shown in FIGS. 7D and 7E, the older mice (6 month old) had significantly smaller muscle lesions, reduced chemokine secretion, but had a much higher tissue bacterial load compared to young (10 week old) mice. In older mice, PVL had no impact on tissue injury or neutrophil recruitment, but had a small albeit significant impact on chemokine secretion (FIGS. 7D and 7E). These findings are consistent with clinical reports that older individuals mount a more limited immune response to bacterial challenge [19], and have less severe pathology associated with PVL [2, 3].

### Contribution of PVL to Infection in Different CA-MRSA Strains

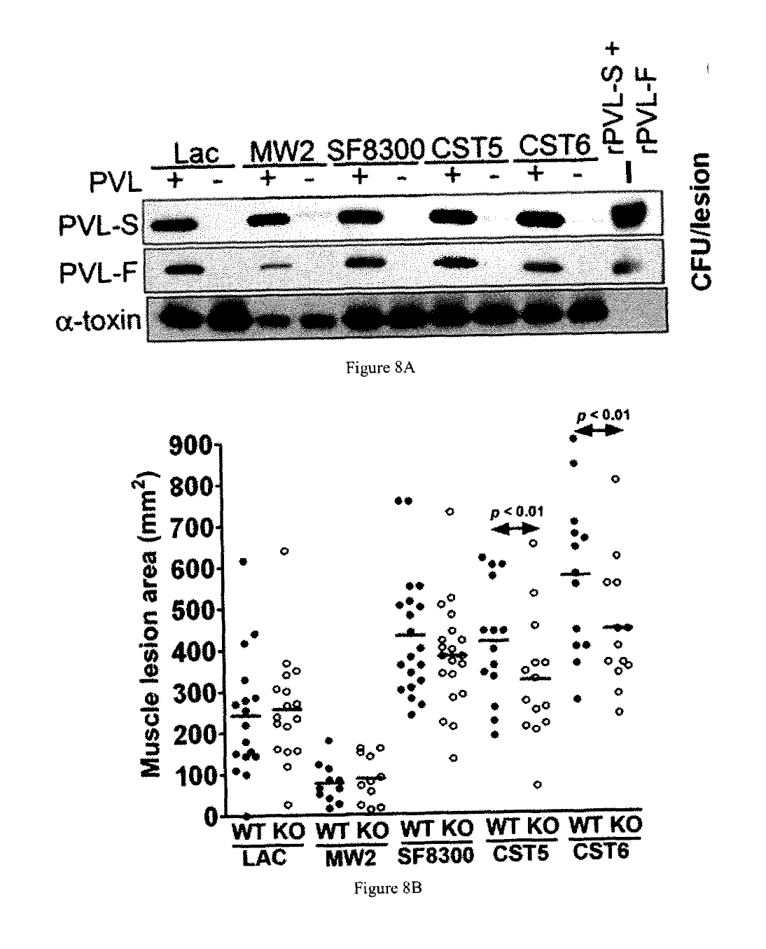

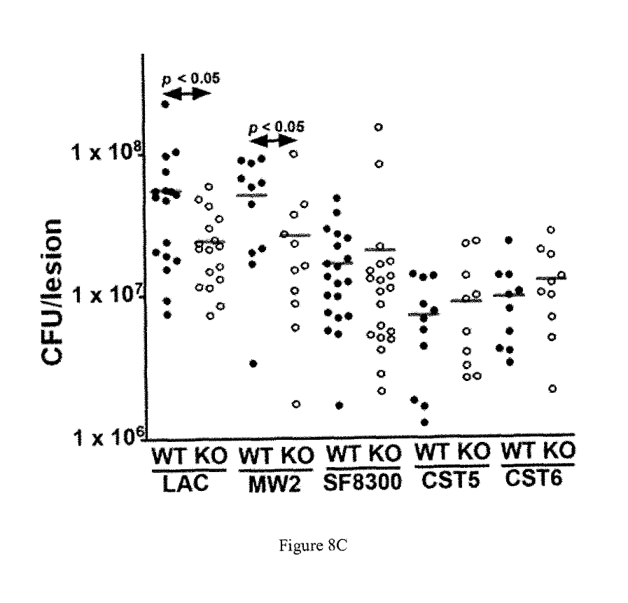

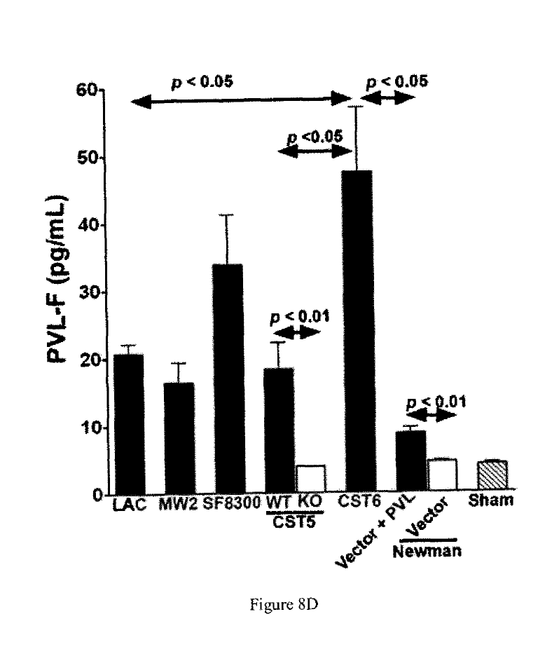

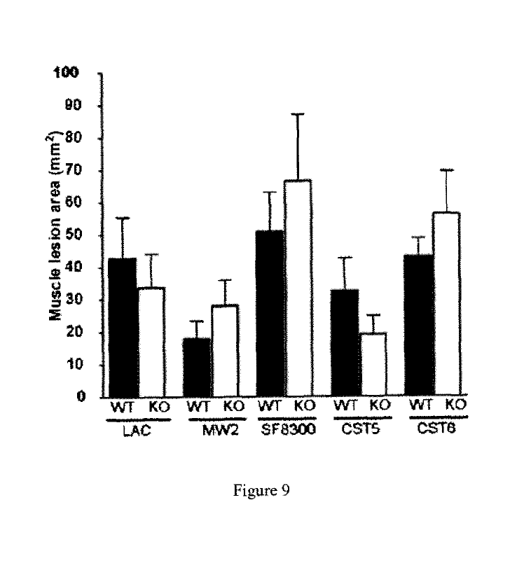

To investigate whether our findings could be generalized to other CA-MRSA strains, we tested the virulence function of PVL using three other pairs of isogenic CA-MRSA with or without PVL in the CD1 model of severe skin and soft tissue infection. In vitro PVL and α-toxin expression from each of the bacterial strains is shown in FIG. 8A. When injected subcutaneously, CST5 and CST6 induced larger muscle lesions compared to the isogenic PVL knockout strains, but on the background of LAC, MW2, and SF8300, PVL had no impact on lesion size (FIG. 8B). Conversely, PVL conferred a small but significant bacterial survival benefit in the MW2 and LAC backgrounds but not in SF8300, CST5, or CST6 backgrounds (FIG. 8C). When an inoculum of 10⁷ CFU was administered, PVL⁺ and PVL⁻ isogenic strains did not produce differences in muscle lesion size (FIG. 9). CST5 and CST6 differed from the other strains in that they were isolated from necrotizing fasciitis patients and also caused more severe injury in mice. To determine whether differential PVL expression by the different strains contributed to differences in PVL virulence in vivo, mice were infected with each of the WT strains, and PVL protein levels in infected tissues were quantified. Overall there were significant differences in PVL expression between strains, but these differences did not correlate with PVL induced injury (FIG. 8D). Thus the data suggests that factors other than PVL play a critical role in modulating the specific virulence effect attributable to PVL.

### Conclusion

The results revealed in the present subject matter markedly shift the focus in the study of CA-MRSA prevention and curtailment from a merely cytolytic function of PVL to the pro-inflammatory properties of PVL, that our data indicates is the primary determinant of disease pathology. In examining PVL functionality, multiple findings were identified that closely parallel human disease, reconcile previous results, and explain hallmark epidemic features associated with PVL. Viewed from this new perspective, many phenomena that once appeared as loose pieces of the CA-MRSA puzzle can now be unified under a single model, revealing the following important implications.

Shift of Focus from Cytolysis to Inflammation

Numerous PVL researchers have argued that PVL could not have a major role in human diseases, pointing to the fact that PVL is a relatively weak toxin compared to other S. aureus toxins and therefore its contribution to infections would be relatively minor [4]. The present subject matter demonstrates that PVL induces inflammation in vivo, thus shifting the focus away from the problematic cytolysis model that has paralyzed the field. In the present subject matter, induction of inflammation by PVL is achieved at a more physiologic dose, and therefore represents a plausible primary mechanism for PVL virulence function.

### Why CA-MRSA Targets the Young and Healthy

One hallmark feature of PVL-linked diseases—reminiscent of the epidemiology of 1918 Spanish flu—is the paradoxical targeting of young and healthy hosts stricken with either necrotizing pneumonia or funrunculosis. In both CA-MRSA and Spanish Flu, individuals with robust immune systems suffer from increased disease severity. In the case of the Spanish Flu, this increased disease severity in healthy individuals is caused by an overly exuberant immune response [13]. Similarly, we find that PVL induced greater inflammation, and this was linked to more severe pathology. Further, PVL effect was most prominent in young mice and strains of mice that were most effective in immune clearance of MRSA, and not significant in older mice and strains of mice that had a more limited response to MRSA. These findings directly parallel human epidemiologic findings, and point to a primary function of PVL as provocateur of inflammation.

### Explanation of Spider Bite-Like Skin Lesions

A widely recognized feature of CA-MRSA skin infections is the spider bite like skin lesions, which practitioners have been taught to recognize when evaluating skin infections likely caused by CA-MRSA. These PVL-linked lesions frequently present with accumulations of PMN (abscesses) and require surgical drainage [20]. The increased incidence of abscesses can be readily explained by increased secretion of chemokines, neutrophil recruitment and inflammation, all of which are characteristics that have been demonstrated in the present subject matter.

### Explanation of Increased Incidence of Deep Tissue Injury

PVL is commonly associated with severe deep tissue infections such as necrotizing fasciitis and myositis [10, 11]. Accordingly, our study shows that PVL does not contribute to skin lesion, but does cause more severe muscle injury.

### Implications for Treatment of CA-MRSA Diseases

We showed that antibody to PVL can ameliorate disease severity due to PVL. Further, our study suggests that modulating the inflammatory response might prove beneficial in treating PVL-associated infections.

In summary, the present subject matter represents a paradigm shift from the current understanding of CA-MRSA pathogenesis generating enormous interest and discussion among microbiologists, epidemiologists, immunobiologists, the general scientific and medical community, as well as the increasingly alarmed general public. The present disclosures and implications stated herein are critical to the understanding and treatment of MRSA, and advance the field forward to focus on detailed mechanisms of PVL virulence, promoting novel and effective approaches to curing the CA-MRSA epidemic.

Various embodiments of the subject matter are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the present subject matter known to the applicant at this time of filing the application has been presented and is intended for the purposes of illustration and description. The present description is not intended to be exhaustive nor limit the subject matter to the precise form disclosed and many modifications and variations are possible in the light of the above teachings. The embodiments described serve to explain the principles of the subject matter and its practical application and to enable others skilled in the art to utilize the present subject matter in various embodiments and with various modifications as are suited to the particular use contemplated. Therefore, it is intended that the present subject matter not be limited to the particular embodiments disclosed for carrying out the present subject matter.

While particular embodiments of the present subject matter have been shown and described, it will be obvious to those skilled in the art that, based upon the teachings herein, changes and modifications may be made without departing from the present subject matter and its broader aspects and, therefore, the appended claims are to encompass within their scope all such changes and modifications as are within the true spirit and scope of the present subject matter. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.)

TABLE 1
Strains and plasmids used in this study

| | | Reference/ |
|---|---|---|
| Name | Description | Source |
| Bacteria strains | | |
| Staphylococcus | | |
| aureus | | |
| MW2 | Clinical isolate. SCCmec IV, ST1, USA400 | 21 |
| LAC | Clinical isolate. SCCmec IV, ST8, USA300 | 21 |
| SF8300 | Clinical isolate. SCCmec IV, ST8, USA300 | 21 |
| CST5 | Clinical isolate. SCCmec IV, ST8, USA300 | 22 |
| CST6 | Clinical isolate. SCCmec IV, ST8, USA300 | 22 |
| RN4220 | Accepts foreign DNA (r⁻) | 23 |
| Newman | Laboratory strain | Goetz, F. |
| CST151 | CST5, luk-PV 2 stop codons-erm^{R} | This study |
| CST153 | CST6, luk-PV 2 stop codons-erm^{R} | This study |
| BD0295 | LAC, luk-PV::spec^{R} | 21 |
| BD0297 | MW2, luk-PV::spec^{R} | 21 |
| BD0299 | SF8300, luk-PV::spec^{R} | This study |
| CST176 | CST5, pDT144 | This study |
| CST178 | CST151, pDT144 | This study |
| CST181 | CST151, pDT145 | This study |
| Newman | pDCErm | This study |
| Newman | pDCErm + PVL | This study |
| Plasmids | | |
| pDT144 | 3.9 kb pCR2.1 BamHI fragment in pDL278 | This study |
| pDT145 | pDT144 + 2.2 kb PVL DNA fragment | This study |
| pDCErm | Shuttle vector | 24 |
| pDCErm + | 2.2 kb PVL expression fragment in pDCErm | This study |
| PVL | | |
| pCR2.1 | PCR cloning vector | Invitrogen |
| pET151 | Expression vector | Invitrogen |
| pDL278 | Shuttle vector | 25 |
| pET151LukS | rLukS expression vector | This study |
| pET151LukF | rLukF expression vector | This study |

LAC, Los Angeles County clone;
MLST, multilocus sequence type;
SSC, staphylococcal cassette chromosome;
ST, sequence type

### REFERENCES

1. Murray et al. Eds, Manual of Clinical Microbiology, 7th Ed., ASM Press, Washington, D.C. (1999).
2. Gillet, Y. et al. Association between Staphylococcus aureus strains carrying gene for Panton-Valentine leukocidin and highly lethal necrotising pneumonia in young immunocompetent patients. Lancet 359, 753-9 (2002).
3. Yamasaki, O. et al. The association between Staphylococcus aureus strains carrying panton-valentine leukocidin genes and the development of deep-seated follicular infection. Clin Infect Dis 40, 381-5 (2005).
4. Bubeck Wardenburg, J., Bae, T., Otto, M., Deleo, F. R. & Schneewind, O. Poring over pores: alpha-hemolysin and Panton-Valentine leukocidin in Staphylococcus aureus pneumonia. Nat Med 13, 1405-6 (2007).
5. Voyich, J. M. et al. Is Panton-Valentine leukocidin the major virulence determinant in community-associated methicillin-resistant Staphylococcus aureus disease? J Infect Dis 194, 1761-70 (2006).
6. Labandeira-Rey, M. et al. Staphylococcus aureus Panton-Valentine leukocidin causes necrotizing pneumonia. Science 315, 1130-3 (2007).
7. Diep, B. A. & Otto, M. The role of virulence determinants in community-associated MRSA pathogenesis. Trends Microbiol (2008).
8. Boyle-Vavra, S. & Daum, R. S. Community-acquired methicillin-resistant Staphylococcus aureus: the role of Panton-Valentine leukocidin. Lab Invest 87, 3-9 (2007).
9. Gordon, R. J. & Lowy, F. D. Pathogenesis of methicillin-resistant Staphylococcus aureus infection. Clin Infect Dis 46 Suppl 5, S350-9 (2008).
10. Miller, L. G. et al. Necrotizing fasciitis caused by community-associated methicillin-resistant Staphylococcus aureus in Los Angeles. N Engl J Med 352, 1445-53 (2005).
11. Pannaraj, P. S., Hulten, K. G., Gonzalez, B. E., Mason, E. O., Jr. & Kaplan, S. L. Infective pyomyositis and myositis in children in the era of community-acquired, methicillin-resistant Staphylococcus aureus infection. Clin Infect Dis 43, 953-60 (2006).
12. Loo, Y. M. & Gale, M., Jr. Influenza: fatal immunity and the 1918 virus. Nature 445, 267-8 (2007).
13. Kash, J. C. et al. Genomic analysis of increased host immune and cell death responses induced by 1918 influenza virus. Nature 443, 578-81 (2006).
14. Konig, B., Prevost, G., Piemont, Y. & Konig, W. Effects of Staphylococcus aureus leukocidins on inflammatory mediator release from human granulocytes. J Infect Dis 171, 607-13 (1995).
15. Stryjewski, M. E. & Chambers, H. F. Skin and soft-tissue infections caused by community-acquired methicillin-resistant Staphylococcus aureus. Clin Infect Dis 46 Suppl 5, S368-77 (2008).
16. Konig, B. et al. Activation of human effector cells by different bacterial toxins (leukocidin, alveolysin, and erythrogenic toxin A): generation of interleukin-8. Infect Immun 62, 4831-7 (1994).
17. Darville, T., Andrews, C. W., Jr., Sikes, J. D., Fraley, P. L. & Rank, R. G. Early local cytokine profiles in strains of mice with different outcomes from chlamydial genital tract infection. Infect Immun 69, 3556-61 (2001).
18. Mosmann, T. R. & Coffman, R. L. TH1 and TH2 cells: different patterns of lymphokine secretion lead to different functional properties. Annu Rev Immunol 7, 145-73 (1989).
19. Ginaldi, L. et al. The immune system in the elderly: III. Innate immunity. Immunol Res 20, 117-26 (1999).
20. Ruhe, J., et al. Community-Onset Methicillin-Resistant Staphylococcus aureus Skin and Soft-Tissue Infections: Impact of Antimicrobial Therapy on Outcome. Clinical Infectious Diseases 44:6, 777-784 (2007).
21. Voyich, J. M. et al. Is Panton-Valentine leukocidin the major virulence determinant in community-associated methicillin-resistant Staphylococcus aureus disease? J Infect Dis 194, 1761-70 (2006).
22. Miller, L. G. et al. Necrotizing fasciitis caused by community-associated methicillin-resistant Staphylococcus aureus in Los Angeles. N Engl J Med 352, 1445-53 (2005).
23. Kreiswirth, B. N. et al. The toxic shock syndrome exotoxin structural gene is not detectably transmitted by a prophage. Nature 305, 709-12 (1983).
24. Jeng, A. et al. Molecular genetic analysis of a group A Streptococcus operon encoding serum opacity factor and a novel fibronectin-binding protein, SfbX. J Bacteriol 185, 1208-17 (2003).
25. LeBlanc, D. J. & Lee, L. N. Replication functions of pVA380-1. in Genetics and molecular biology of streptococci, enterococci, and lactococc (eds. Dunny, G. M., Cleary, P. P. & McKay, L. L.) 224-227 (American Society for Microbiology, Washington, D.C., 1991).
26. Bocchini C E, Hulten K G, Mason D O Jr. Gonzalez B E, Hammerman W A, et al., Panton-Valentine leukocidin genes are associated with enhanced inflammatory response and local disease in acute hematogenous Staphylococcus aureus osteomyelitis in children. Pediatrics 117: 433-440. (2006).
27. Dohin B, Gillet Y, Kohler R, Lina G, Vandenesch F. et al., Pediatric bone and joint infections caused by Panton-Valentine leukocidin-positive Staphylococcus aureus. Pediatr Infect Dis J 26: 1042-1048 (2007).
28. Dufour P, Gillet Y, Bes M, Lina G, Vandenesch F, et al., Community acquired methicillin-resistant Staphylococcus aureus infections in France: emergence of a single clone that produces Panton-Valentine leukocidin. Clin. Infect Dis 35: 819-824 (2002).
29. Robinson D A, Kearns A M, Holmes A, Morrison D, Grundmann H, et al., Re-emergence of early pandemic Staphylococcus aureus as a community acquired meticillin-resistant clone. Lancet 365: 1256-1258 (2005).
30. Lina G, Piemont Y, Godail-Gamot F, Bes M, Peter M O, et al., Involvement of Panton-Valentine leukocidin producing Staphylococcus aureus in primary skin infections and pneumonia. Clin. Infect. Dis. 29: 1128-1132 (1990).
31. Bubeck Wardenburg J, Palazzolo-Ballance A M, Otto M, Schneewind O, DeLeo F R, Panton-Valentine leukocidin is not a virulence determinant in murine models of community-associated methicillin-resistant Staphylococcus aureus disease. J. Infect. Dis. 198: 1166-1170. PVL Promotes Myositis PLoS ONE|www.plosone.org 9 Jul. 2009|Volume 4|Issue 7|e6387 (2008).
32. Brown E L, Dumitrescu O, Thomas D, Badiou C, Koers E M, et al., The Panton-Valentine leukocidin vaccine protects mice against lung and skin infections caused by Staphylococcus aureus USA300. Clin, Microbiol. Infect. 15: 156-164 (2009).
33. Montgomery C P, Daum R S, Transcription of inflammatory genes in the lung after infection with community-associated methicillin-resistant Staphylococcus aureus: a role for panton-valentine leukocidin. Infect. Immun. 77: 2159-2167 (2009).
34. Szmigielski S, Prevost G, Monteil H, Colin D A, Jeljaszewicz J, Leukocidal toxins of staphylococci. Zentralbl Bakteriol 289: 185-201 (1999).
35. Badiou C, Dumitrescu O, Croze M, Gillet Y, Dohin B, et al., Panton-Valentine leukocidin is expressed at toxic levels in human skin abscesses. Clin. Microbial. Infect. 14: 1180-1183 (2008).
36. Pannaraj P S, Hulten K G, Gonzalez B E, Mason E O Jr, Kaplan S L, Infective pyomyositis and myositis in children in the era of community-acquired, methicillin-resistant Staphylococcus aureus infection. Clin Infect Dis 43: 953-960 (2006).
37. Konig B, Prevost G, Piemont Y, Konig W., Effects of Staphylococcus aureus leukocidins on inflammatory mediator release from human granulocytes. J Infect Dis 171: 607-613 (1995).
38. Stryjewski M E, Chambers H F, Skin and soft-tissue infections caused by community-acquired methicillin-resistant Staphylococcus aureus. Clin Infect Dis. 46 Suppl 5: S368-377 (2008).
39. Bunce C, Wheeler L, Reed 0, Musser J, Barg N., Murine model of cutaneous infection with gram-positive cocci. Infect. Immun. 60: 2636-2640 (1992).
40. Shrout P E, Fleiss J. L., Intraclass correlations: uses in assessing rater reliability. Psychol. Bull. 86: 420-428 (1979).
41. Girish V, Vijayalakshmi A., Affordable image analysis using NIH Image/ImageJ. Indian J. Cancer 41:47 (2004).

What is claimed is:

1. A method of treating a MRSA infection in a subject, comprising:
providing an anti-inflammatory drug; and
administering the anti-inflammatory drug to the subject by a route of delivery.

2. The method of claim 1, wherein the anti-inflammatory drug is selected from the group consisting of corticosteroids, cyclooxygenase-inhibitors, ibuprofen, diclofenac, licofelone, aspirin, celecoxib, ketaprofen, piroxicam, sulindac, etoricoxib, fenbufen, fenoprofen, floctafenine, flur-biprofen, tiaprofenic acid, azapropazone, diflunisal, etodolac, lumiracoxib, indomethacin (indometacin), ketorolac, meclofenamate, mefenamic, meloxicam, nabumetone, naproxen, oxaprozin, piroxicam, phenylbutazone, sunlindac, rofecoxib, tenoxicam, tolfenamic acid, tolmetin, valdecoxib, nimesulide, steroids, derivatives thereof, analogs thereof, and combinations thereof.

3. The method of claim 1, wherein a condition associated with the MRSA infection is ameliorated, and said condition is selected from the group consisting of necrotic tissue injury, necrotized fasciitis, muscle inflammation, deep tissue necrosis, necrotizing pneumonia, furunculosis, and combinations thereof.

4. The method of claim 1, wherein the route of delivery is selected from the group consisting of non-invasive peroral, topical, transmucosal, inhalation, parenteral, and combinations thereof.

5. A method of treating a Pantone-Valentine leukocidin associated infection in a subject, comprising:
providing an anti-inflammatory drug; and
administering the anti-inflammatory drug to the subject by a route of delivery.

6. The method of claim 5, wherein the anti-inflammatory drug is selected from the group consisting of corticosteroids, cyclooxygenase-inhibitors, ibuprofen, diclofenac, licofelone, aspirin, celecoxib, ketaprofen, piroxicam, sulindac, etoricoxib, fenbufen, fenoprofen, floctafenine, flur-biprofen, tiaprofenic acid, azapropazone, diflunisal, etodolac, lumiracoxib, indomethacin (indometacin), ketorolac, meclofenamate, mefenamic, meloxicam, nabumetone, naproxen, oxaprozin, piroxicam, phenylbutazone, sunlindac, rofecoxib, tenoxicam, tolfenamic acid, tolmetin, valdecoxib, nimesulide, steroids, derivatives thereof, analogs thereof, and combinations thereof.

7. The method of claim 5, wherein a condition associated with the Pantone-Valentine leukocidin associated infection is ameliorated, and said condition is selected from the group consisting of necrotic tissue injury, necrotized fasciitis, muscle inflammation, deep tissue necrosis, necrotizing pneumonia, furunculosis, and combinations thereof.

8. The method of claim 5, wherein the route of delivery is selected from the group consisting of non-invasive peroral, topical, transmucosal, inhalation, parenteral, and combinations thereof.


SEQUENCE LISTING (10 sequences)

SEQ ID NO: 1 - DNA, 22 bp, Artificial Sequence
  Staphylococcus aureus
gaaaggaaatgatttttaggtc

SEQ ID NO: 2 - DNA, 21 bp, Artificial Sequence
  Staphylococcus aureus
gacctaaaaatcatttccttc

SEQ ID NO: 3 - DNA, 23 bp, Artificial Sequence
  Staphylococcus aureus
aatattctattggaaaggccacc

SEQ ID NO: 4 - DNA, 23 bp, Artificial Sequence
  Staphylococcus aureus
ctcaatattgttatcagctttag

SEQ ID NO: 5 - DNA, 35 bp, Artificial Sequence
  Staphylococcus aureus
ctgaccgcggtatgacggcgcatattgtatcaatg

SEQ ID NO: 6 - DNA, 35 bp, Artificial Sequence
  Staphylococcus aureus
gccaggatcccacgtcaattaagacgtggttaccc

SEQ ID NO: 7 - DNA, 25 bp, Artificial Sequence
  Staphylococcus aureus
caccgaatctaaagctgataacaat

SEQ ID NO: 8 - DNA, 27 bp, Artificial Sequence
  Staphylococcus aureus
tcaattatgtcctttcactttaatttc

SEQ ID NO: 9 - DNA, 24 bp, Artificial Sequence
  Staphylococcus aureus
caccgctcaacatatcacacctgt

SEQ ID NO: 10 - DNA, 26 bp, Artificial Sequence
  Staphylococcus aureus
ttagctcataggatttttttccttag